SEQUENCE LISTING (16 sequences)

SEQ ID NO: 1 - DNA, 2022 bp, Artificial Sequence
  GGPP synthase, crtE
atgacggcattggcatactaccagatccacctcatctacaccctccccatcctcggtctcctcggtctgcttacaagccccatcctcacaaagttcgacatctacaagatctcgatcctcgtgttcatcgcgttctccgccacgacgccctgggacagctggatcatccgaaacggcgcatggacgtaccctagcgcagaatctggtcaaggtgttttcggcacgttcctggacgttccctacgaggagtacgcattcttcgtgatccagacggtgatcactggcctggtctacgtcctcgcaacacgacacctcctcccatctctggcattgcctaagacacgttcctctgctctctctctggctctcaaggcactcatccctttgcctatcatctacctgttcaccgcccacccatccccatctccagacccattggtcaccgaccattacttctacatgcgggctctgtccctcctcatcacccctcctactatgctgttggctgctttgtccggtgagtacgctttcgactggaagtcgggaagagctaagtccaccatcgctgctatcatgatccccaccgtgtacctcatctgggtggactacgtcgctgtgggacaagactcttggtcgattaacgacgagaagattgtcggctggcgactgggaggagtcctgcctattgaggaggctatgttcttcctgctcaccaacctgatgattgtgctcggcctctccgcctgcgaccatacacaggctctctacctgttgcacggtagaaccatttacggcaacaagaagatgccgtcgtccttccccctgattaccccccctgtgctgtctctcttcttcagctcccgaccttactcgtcccagcctaagcgagacttggagttggctgtgaagctgctggaggagaagtctcgtagcttcttcgtcgcttccgctggattcccttctgaggtcagagagcgactcgttggtttgtacgcgttttgccgagtgaccgacgacctcattgactcgcctgaggttagctctaaccctcacgctacaattgacatggtgtccgactttctgaccctgctctttggaccgccgctgcacccttctcagcctgacaagattctgtcctcgcctctcctccctccttctcacccctctagacccactggaatgtaccccttgcccccaccaccgtctttgtctcccgctgaattggttcaatttcttacagagcgtgttcccgttcaataccattttgcctttcgtctgctggccaagctgcagggtctgattccgcgttacccgctggatgagctgcttcggggttataccaccgatctgatttttcccctgtccaccgaggctgttcaggcgagaaagacccccattgagaccaccgccgatcttcttgactacggtctttgtgtggccggatctgttgccgaacttctggtttatgtgtcctgggcgtctgcgccctctcaggttcccgcgactattgaagagagagaggccgttcttgtggcctctagagaaatgggaaccgcccttcagcttgtgaatattgcccgagacattaagggcgatgccactgagggacgattttatctgcccctgagcttttttggactgcgggatgagtcgaagctggccattcccactgattggactgagccgcgaccccaggattttgataagctgctgtcgctgagcccctcgtcgactctgcccagctccaatgcctcggaatcctttcgatttgagtggaagacttattcgctgccccttgtggcctatgccgaggatcttgccaaacattcgtataaaggcattgatcgacttcccactgaagtccaggccggcatgcgagccgcctgtgccagctatcttcttattggacgggaaattaaagtcgtctggaaaggcgatgtgggagaacgacggactgtggccggctggcggcgagtccgaaaagtcctttccgtcgtcatgtcgggctgggaaggccagtaa

SEQ ID NO: 2 - DNA, 1131 bp, Artificial Sequence
  Phytoene synthase, crtYB
atggactacgcaaacatcctcaccgcaatcccattggagttcaccccacaggatgatatcgtgctgttggagccttaccactacctcggtaagaaccctggtaaggagatccgttctcagctcatcgaggcattcaactactggctggatgtcaagaaggaggacctggaggtcatccagaacgtcgttggaatgttgcacacggcttctttgctcatggacgatgtggaggattcttcggtgttgcgtagaggttctcctgttgctcacctcatctacggtatccctcagacaatcaacaccgctaactacgtgtacttcctggcgtaccaggagatcttcaagctgcgacctacgcctatccccatgcccgttatccccccgtcttctgcttctttgcaatcttctgtttcttccgcttcttcttcctccagcgctagctccgaaaacggtggaacaagcacacccaattcccaaatccccttctccaaggatacatacctcgataaggtgattacggacgagatgctctccctgcacagaggacagggacttgagcttttctggagagactcgcttacttgcccgtcggaagaagagtacgttaagatggtgctgggaaagaccggaggccttttccgaattgccgttcgacttatgatggccaagtccgagtgtgacattgacttcgtccagcttgtcaacctgatttccatttactttcagattcgagacgactacatgaacctgcagtcgtcggagtatgcccacaacaagaactttgccgaggacctgaccgagggcaagttttcctttcccaccattcactcgattcataccaaccccagctcgcggctggtgattaacaccctgcagaagaagtccacctcgcccgagattctccatcattgcgtcaactatatgcggactgagactcattcgtttgagtatactcgagaggtgctgaatactctgtccggcgcgctggagcgtgagctcggccgacttcaagaagaatttgccgaagccaatagccggatggacctgggcgacgtcgaaagcgaaggccgaactggcaaaaatgtgaaactggaagccattctcaaaaaactcgccgacattccgctctaa

SEQ ID NO: 3 - DNA, 1749 bp, Artificial Sequence
  desaturase, crtI
atgggtaaggagcaggatcaggataagccgacagctatcatcgtcggttgcggtatcggaggaatcgcaacagctgcacgtctcgcaaaggagggtttccaagttacggttttcgagaagaacgactacagcggcggtcgttgcagcttgatcgagcgtgatggttaccgtttcgaccagggtccatctttgctgttgctgccagatctcttcaagcagaccttcgaagacctgggtgagaagatggaggactgggtggatctgatcaagtgcgagccaaactacgtgtgccacttccacgacgaggagacattcacgctcagcactgatatggctctcctcaagcgagaggttgagcgtttcgagggtaaggacggtttcgatcggttcctctctttcatccaggaggcacaccggcattacgagttggcagttgtgcacgttctccagaagaacttccctggtttcgcagctttcctccgactccagttcatcggtcagatcctggctctccaccctttcgaatctatctggacccgagtttgtcgatacttcaagaccgaccgactgcggcgggttttctctttcgctgtgatgtacatgggccagtctccatactccgctcctggtacttactcgctcctccagtacactgagctcactgagggaatctggtaccctagaggaggattctggcaggttcctaacaccctcctccagatcgttaagcgaaacaacccgtctgctaagttcaacttcaacgcgccggtctcccaggttttgctgtcgcctgctaaggatcgagctactggagttcgactggagtctggagaggagcatcacgctgatgtcgtcatcgtgaacgctgatttggtgtacgcttctgagcacctcatccctgatgacgctcggaacaagatcggacagttgggagaggtgaagagatcttggtgggcggatctcgtgggaggaaaaaagttgaagggatcgtgttcgtccctgtctttttactggtccatggatcgaattgtggacggcctgggcggacacaacatttttctggcggaggactttaagggcagctttgacaccatttttgaggaactgggcctgcccgccgacccttctttttacgtcaacgtgccctcgcgaattgacccctccgccgcccctgaaggaaaagacgccattgtcattctggtcccttgtggacatattgacgcctctaacccccaggactataacaagctggtcgcccgagccagaaaatttgtgattcacaccctgtccgcgaagctgggactgcccgactttgaaaagatgattgtggccgaaaaggtccacgacgccccctcctgggaaaaagaatttaacctgaaggacggctccattctgggcctggcccataattttatgcaagtgctgggctttcgaccgagcacgcgacatcccaagtatgacaagctgttttttgtcggcgcctccacccatcccggaactggcgtgcccattgtccttgccggcgccaaacttaccgccaatcaagtccttgaatcgtttgacagatcccccgcccccgaccccaatatgtcgctttccgtgccctatggcaaaccccttaaatcgaatggcaccggcattgactcgcaagtccaacttaagtttatggacctggaaagatgggtgtatctgcttgtccttcttattggcgccgtcattgccagaagcgtgggcgtgcttgccttttaa

SEQ ID NO: 4 - DNA, 957 bp, Halobacterium salinarum
gaggacgccctgcacgcggtccatccacgtgaccacggcgacgtgtggcagggtgagcacggcgatcaacacgaggtagagtgcggccccggattcgagcgtggtcggcgggttgggtaccgcccaccacagcacgccgcccaacgcgagcgcggccgccgtcatcggcgcggcctccacgccgaaccgggccagcggtccgaggatgtcgcccgcgcgcagcgacgggtggaccgagccgtcgacggcgatggcgcgcgcgacgtgccggaccgagtgccagacgcagaagtagacgccgacggcgaacaccggcggcacgacgaagaagaacacccacaacaggagggtttcggcggcgtcgacgcgccacgcgccgggggagtgggtgcggcgtctgcccgccgcgagcaccgcgacggtcgcggctgcgaacgcgacgcccagccacaggcggacgcgcgggtcgaacaccgcgaggtcgccgacggagccgccgaacggcgcggcgaacgcgtcgacgacgctgcggtaccgctccgggaacccgagcagcggcacgagcatcgggaggccaccccggatcaggaccgtcgccgcgcgccgcgggcgcgtatcgaggtagtcgacgtcgaggaagtcgagcagcgggtagaggtcgccctgcccccagtgcagccacgtgatcgcgacgaacgcgaacgcagcgggaacgggcgcgaaaaaccacgcggcggcgtagccaccaccgagcacgaggtagaggacgccgacgaccgccagccaccgcacggtgacggtgcccgtgaccgcgcggggcaacgcgaggtagtcgatcgcgccgtgtggcatgccgaacaccaccgcgctggcgaccagcggggcgtaccgtgcggtcggcgaaagcgacacgccagtgatcgcgggcagggcagcgatgctgatcgccacccagcc

SEQ ID NO: 5 - DNA, 825 bp, Artificial Sequence
  predicted Brp-like protein Blh
atgggcttgatgttaattgattggtgtgctttagcattggttgtgtttattggtttgccacatggtgccttagatgctgctatttctttttcaatgatttcttcagcaaagagaattgctagattagcaggaatactattaatttacctgttgttagcaaccgcattttttttaatttggtatcaattaccagcattttctcttcttatttttcttttgataagcataatccattttggaatggctgatttcaatgcatccccaagtaaacttaagtggcctcatattattgcacatggcggcgttgttactgtttggttgccgcttatccaaaaaaatgaagttacgaagctattttcaatattaacaaatggtccaactcccattttatgggacatactattgatattttttttatgttggagcataggagtatgtcttcatacctatgaaactttacgttctaaacattataatatcgcctttgaacttattggattaatttttctagcctggtatgcacccccactcgttacttttgccacatacttctgctttatccacagcagacgtcactttagttttgtttggaaacagttacagcatatgagttcaaaaaaaatgatgataggtagtgccattattttatcttgtacgagctggttgataggcggaggaatatattttttcctcaattcgaaaatgattgccagtgaagctgctttacaaactgtctttattggtcttgcagctttaacagttcctcacatgatacttatcgactttatatttagaccacactcttccagaattaaaatcaaaaat

SEQ ID NO: 6 - DNA, 807 bp, Escherichia coli
atgactcataaagcaacggagatcctgacaggtaaagttatgcaaaaatcggtcttaattaccggatgttccagtggaattggcctggaaagcgcgctcgaattaaaacgccagggttttcatgtgctggcaggttgccggaaaccggatgatgttgagcgcatgaacagcatgggatttaccggcgtgttgatcgatctggattcaccagaaagtgttgatcgcgcagccgacgaggtgatcgccctgaccgataattgtctgtatgggatctttaacaatgccggattcggcatgtatggccccctttccaccatcagccgtgcgcagatggaacagcagttttccgccaactttttcggcgcacaccagctcaccatgcgcctgttacccgcgatgttgccgcacggtgaagggcgtattgtgatgacctcatcggtgatgggattaatctccacaccgggtcgtggcgcttacgcggccagtaaatatgcgctggaggcgtggtcagacgcgctgcgcatggagctgcgccgcagcggaattaaagtcagcctgatcgaacccggtcccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtaacgctggtgacctgggcggtaatggtgcttaagcgcctgctgccggggcgcgtgatggacaaaatattgcagggg

SEQ ID NO: 7 - protein, 673 aa, Artificial Sequence
  GGPP synthase, crtE
MTALAYYQIHLIYTLPILGLLGLLTSPILTKFDIYKISILVFIAFSATTPWDSWIIRNGAWTYPSAESGQGVFGTFLDVPYEEYAFFVIQTVITGLVYVLATRHLLPSLALPKTRSSALSLALKALIPLPIIYLFTAHPSPSPDPLVTDHYFYMRALSLLITPPTMLLAALSGEYAFDWKSGRAKSTIAAIMIPTVYLIWVDYVAVGQDSWSINDEKIVGWRLGGVLPIEEAMFFLLTNLMIVLGLSACDHTQALYLLHGRTIYGNKKMPSSFPLITPPVLSLFFSSRPYSSQPKRDLELAVKLLEEKSRSFFVASAGFPSEVRERLVGLYAFCRVTDDLIDSPEVSSNPHATIDMVSDFLTLLFGPPLHPSQPDKILSSPLLPPSHPSRPTGMYPLPPPPSLSPAELVQFLTERVPVQYHFAFRLLAKLQGLIPRYPLDELLRGYTTDLIFPLSTEAVQARKTPIETTADLLDYGLCVAGSVAELLVYVSWASAPSQVPATIEEREAVLVASREMGTALQLVNIARDIKGDATEGRFYLPLSFFGLRDESKLAIPTDWTEPRPQDFDKLLSLSPSSTLPSSNASESFRFEWKTYSLPLVAYAEDLAKHSYKGIDRLPTEVQAGMRAACASYLLIGREIKVVWKGDVGERRTVAGWRRVRKVLSVVMSGWEGQ

SEQ ID NO: 8 - protein, 376 aa, Artificial Sequence
  Phytoene synthase, crtYB
MDYANILTAIPLEFTPQDDIVLLEPYHYLGKNPGKEIRSQLIEAFNYWLDVKKEDLEVIQNVVGMLHTASLLMDDVEDSSVLRRGSPVAHLIYGIPQTINTANYVYFLAYQEIFKLRPTPIPMPVIPPSSASLQSSVSSASSSSSASSENGGTSTPNSQIPFSKDTYLDKVITDEMLSLHRGQGLELFWRDSLTCPSEEEYVKMVLGKTGGLFRIAVRLMMAKSECDIDFVQLVNLISIYFQIRDDYMNLQSSEYAHNKNFAEDLTEGKFSFPTIHSIHTNPSSRLVINTLQKKSTSPEILHHCVNYMRTETHSFEYTREVLNTLSGALERELGRLQEEFAEANSRMDLGDVESEGRTGKNVKLEAILKKLADIPL

SEQ ID NO: 9 - protein, 582 aa, Artificial Sequence
  desaturase, crtI
MGKEQDQDKPTAIIVGCGIGGIATAARLAKEGFQVTVFEKNDYSGGRCSLIERDGYRFDQGPSLLLLPDLFKQTFEDLGEKMEDWVDLIKCEPNYVCHFHDEETFTLSTDMALLKREVERFEGKDGFDRFLSFIQEAHRHYELAVVHVLQKNFPGFAAFLRLQFIGQILALHPFESIWTRVCRYFKTDRLRRVFSFAVMYMGQSPYSAPGTYSLLQYTELTEGIWYPRGGFWQVPNTLLQIVKRNNPSAKFNFNAPVSQVLLSPAKDRATGVRLESGEEHHADVVIVNADLVYASEHLIPDDARNKIGQLGEVKRSWWADLVGGKKLKGSCSSLSFYWSMDRIVDGLGGHNIFLAEDFKGSFDTIFEELGLPADPSFYVNVPSRIDPSAAPEGKDAIVILVPCGHIDASNPQDYNKLVARARKFVIHTLSAKLGLPDFEKMIVAEKVHDAPSWEKEFNLKDGSILGLAHNFMQVLGFRPSTRHPKYDKLFFVGASTHPGTGVPIVLAGAKLTANQVLESFDRSPAPDPNMSLSVPYGKPLKSNGTGIDSQVQLKFMDLERWVYLLVLLIGAVIARSVGVLAF

SEQ ID NO: 10 - protein, 338 aa, Halobacterium salinarum
MALTPLTARARRTLARPALALGWVAISIAALPAITGVSLSPTARYAPLVASAVVFGMPHGAIDYLALPRAVTGTVTVRWLAVVGVLYLVLGGGYAAAWFFAPVPAAFAFVAITWLHWGQGDLYPLLDFLDVDYLDTRPRRAATVLIRGGLPMLVPLLGFPERYRSVVDAFAAPFGGSVGDLAVFDPRVRLWLGVAFAAATVAVLAAGRRRTHSPGAWRDAAETLLLWVFFFVVPPVFAVGVYFCVWHSVRHVARAIAVDGSVHPSLRAGDILGPLARFGVEAAPMTAAALALGGVLWAVPNPPTTLESGAALYLVLIAVLTLPHVAVVTWMDRVQGVL

SEQ ID NO: 11 - protein, 275 aa, Artificial Sequence [sequence omitted]

SEQ ID NO: 12 - protein, 269 aa, Escherichia coli
MTHKATEMLTGKVMQKSVTITGCSSGMGLESALETKRQGFHVLAGCRKPDDVERMNSMGFTGVLIDLDSPESVDRAADEVIATTDNCLYGIFNNAGFGMYGPTSTISRAQMEQQFSANFFGAHQLTMRTLPAMLPHGEGRIVMTSSVMGLISTPGRGAYAASKYALEAWSDALRMELRHSGIKVSTMEPGPIRTRFTDNVNQTQSDKPVENPGMAARFTLGPEAVVDKVRHAFISEKPKMRYPVTTVTWAVMVTKRLLPGRVMDKILQG

SEQ ID NO: 13 - DNA, 70 bp, Artificial Sequence
  BCMO primer-F
atcgtccttgtatggaagtatcaaaggggacgttcttcacctccttggaaccagtcacgacgttgtaaaa

SEQ ID NO: 14 - DNA, 70 bp, Artificial Sequence
  BCMO primer-R
aatgatttacaatctagtcgcaaaaacaagtacagtgctgacgtcccatcaggtttcccgactggaaagc

SEQ ID NO: 15 - DNA, 69 bp, Artificial Sequence
  ybbO primer-F
aaatccgaacaacagagcatagggtttcgcaaacaaacttaaatatatgcaggtttcccgactggaaag

SEQ ID NO: 16 - DNA, 69 bp, Artificial Sequence
  ybbO primer-R
gtataatctgtatacataatattataggctttaccaacaatggaatttcgccagtcacgacgttgtaaa

(19) United States
(12) Patent Application Publication
(10) Pub. No.: US 2022/0017878 A1
(43) Pub. Date: Jan. 20, 2022

(54) MICROORGANISM FOR PRODUCING BIORETINOL AND METHOD OF PRODUCING BIORETINOL USING THE SAME

(71) Applicant: BIOSPLASH CO., LTD., Gyeonggi-do (KR)

(72) Inventors: Byung Jo Yu, Gyeonggi-do (KR); in Seung Jang, Busan (KR); Ji Yeon Jang, Seoul (KR)

(21) Appl. No.: 17/298,307

(22) PCT Filed: Nov. 29, 2019
(86) PCT No.: PCT/KR2019/016783

§ 371 Date: May 28, 2021

(30) Foreign Application Priority Data
Nov. 30, 2018 (KR) 10-2018-0152617

(51) Int. Cl. C12N 9/04; C12N 9/10; C12N 9/02; C12N 9/16; C12P 23/00

(52) U.S. Cl. CPC C12N 9/0006; C12Y 101/01105; C12Y 205/01032; C12N 9/1085; C12Y 113/11063; C12P 23/00; C12Y 301/07011; C12N 9/16; C12Y 114/19001; C12N 9/0071; C12N 9/0069

(57) ABSTRACT

Provided are a microorganism for producing retinol, in which retinol biosynthetic genes are introduced; and a method of producing retinol, the method including a step of culturing the microorganism. The microorganism of the present invention may have an improved ability to produce retinol, and thus it may be efficiently used in producing retinol. Based on the method of producing retinol, the method including the step of culturing the microorganism, the retinol production efficiency may be improved.

### BACKGROUND OF THE INVENTION

### 1. Field of the Invention

The present invention relates to a microorganism for producing bioretinol, in which beta-carotene and retinol biosynthetic genes are introduced; and a method of producing bioretinol, the method including a step of culturing the microorganism.

### 2. Description of the Related Art

Retinol is a high-value-added raw material that has anti-wrinkle and antioxidant effects. Specifically, retinol and derivatives thereof, which are ingredients that help improve skin wrinkles, are raw materials that provide excellent efficacy in improving wrinkles such that they make up three of the four ingredients listed as raw materials for functional cosmetics in a notification issued by the Ministry of Food and Drug Safety. Therefore, retinol having the above functions may be used as a cosmetic composition having an anti-wrinkle effect.

However, despite such usefulness of retinol, mass production of retinol is not easy. The existing representative method of producing retinol is to produce the final products, retinol and derivatives thereof, through chemical reactions of pentadiene→retinal→retinol after extracting precursors from natural resources. However, the method through such a chemical process has a production cost of 3,500 to 4,000 US dollars per 1 kg, which causes a problem of increasing the production cost of retinol-containing products. Accordingly, in order to achieve more stable production and high yield, a highly efficient retinol production technology through a bio-process is required.

The present inventors have made many efforts to increase the production of retinol, and as a result, they have developed a microorganism capable of more stably producing retinol with high efficiency, and confirmed that the production amount of retinol was increased by culturing the microorganism under various conditions, thereby completing the present invention. When retinol is produced by a yeast fermentation process of the present invention, safety is high because an eco-friendly and safe yeast strain is used. It is also expected to improve price competitiveness, as compared to the existing production technology, because high-efficiency production is possible due to BMCO expression using a multi-copy plasmid.

### SUMMARY OF THE INVENTION

An object of the present invention is to provide a microorganism for producing retinol, in which geranylgeranyl diphosphate synthase (GGPP synthase, crtE), phytoene synthase (crtYB), desaturase (crtl), beta-carotene 15,15′-monooxygenase (β-carotene 15,15′-monooxygenase, BCMO), and retinol dehydrogenase (ybbO) protein activities are enhanced.

Another object of the present invention is to provide a method of producing retinol, the method including a step of culturing the microorganism in a culture medium.

### BRIEF DESCRIPTION OF THE DRAWINGS

FIG. 1 shows the design of biosynthetic metabolic pathways of retinol:

FIG. 2A-C shows SDS-PAGE confirming integration of crtE(A), crtl(B), and crtYB(C) genes;

FIG. 3 shows PCR results confirming integration of BCMO-SR or BCMO-blh gene;

FIG. 4A-B shows colony formation (A) and master colony formation (B) confirming integration of BCMO-SR or BCMO-blh gene;

FIG. 5A-B shows SDS-PAGE confirming integration of BCMO-SR(A) or BCMO-blh(B) gene;

FIG. 6 shows PCR results confirming integration of yybO gene;

FIG. 7A-B shows HPLC/UV chromatographic analysis measuring production amounts of retinal (A) and retinol (B) of a transformed strain of the present invention;

FIG. 8A-B shows an ESI-Mass spectrum graph measuring production amounts of retinol of a wild-type strain (A) and the transformed strain (B) of the present invention;

FIG. 9 shows an HPLC graph measuring production amounts of retinol when BMCO genes were introduced using a single-copy plasmid and a multi-copy plasmid; and

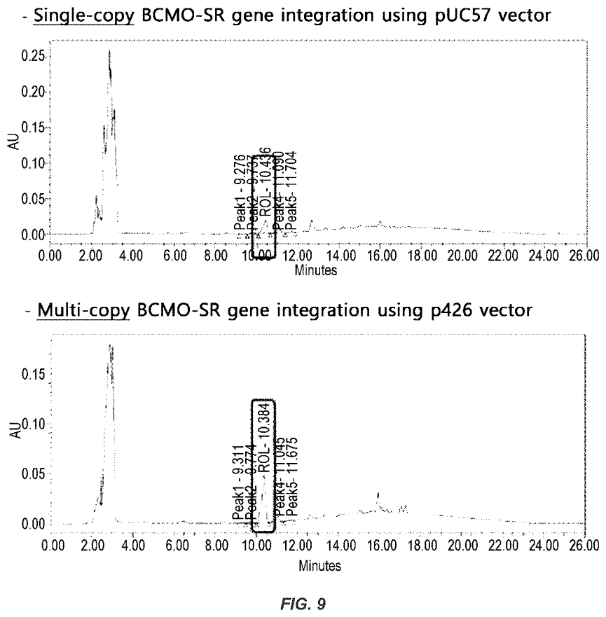

FIG. 10 shows a graph showing a quantitative comparison result of retinol production when the single-copy plasmid or the multi-copy plasmid was used.

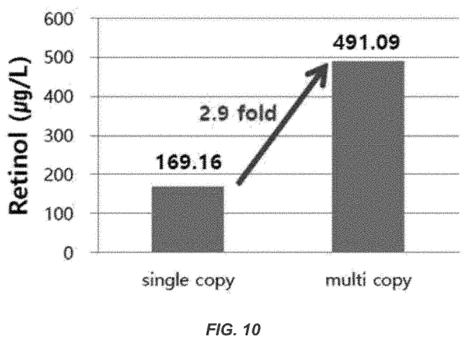

### DETAILED DESCRIPTION OF THE PREFERRED EMBODIMENTS

To achieve the above objects, an aspect of the present disclosure provides a microorganism for producing retinol, in which geranylgeranyl diphosphate synthase (GGPP synthase, crtE), phytoene synthase (crtYB), desaturase (crtl), beta-carotene 15,15′-monooxygenase (β-carotene 15,15′-monooxygenase, BCMO), and retinol dehydrogenase (ybbO) protein activities are enhanced.

Further, the present invention provides use of the microorganism, in which geranylgeranyl diphosphate synthase (GGPP synthase, crtE), phytoene synthase (crtYB), desaturase (crtl), beta-carotene 15,15′-monooxygenase (β-carotene 15,15′-monooxygenase, BCMO), and retinol dehydrogenase (ybbO) protein activities are enhanced, in retinol production.

As used herein, the term “retinol” refers to a type of vitamin A, and retinol has an important role in maintaining original functions of the skin's epidermal cells. Specifically, retinol is the chemical name of vitamin A1, which exists in the intestinal mucosa of animals, and is known to be contained in large amounts in greenish-yellow plants. Retinol is also transformed into retinoic acid as an active form, and allows DNA to express RNA in the cell nuclei present in skin cells to promote cell differentiation, and has efficacy of reducing wrinkles and improving skin elasticity due to its promotion of biosynthesis of collagen, which is a fibrous protein that exists as a fibrous solid between animal cells, and elastin, which is composed of elastic fibers. As described, retinol has been used as a cosmetic ingredient. However, after extracting precursors from natural plant resources, retinol and derivatives thereof, which are final products, are mainly produced through chemical reactions, and such a chemical production method has disadvantages in that purity is low due to high probability of contamination of other impurities, and their production amounts are low to increase the unit cost of production. In contrast, retinol produced using the high-efficiency fermentation production technology of the present invention may be used as a cosmetic composition having effects of preventing skin aging, improving skin elasticity, and improving wrinkles.

As used herein, the term “geranylgeranyl diphosphate synthase (GGPP synthase, crtE)” refers to an enzyme that catalyzes the reversible reaction of the following Reaction Scheme.

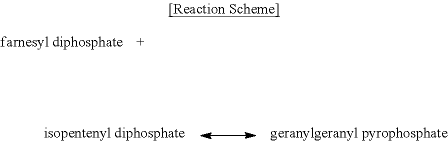

As used herein, the term “phytoene synthase (crtYB)”, which is one of the enzymes involved in carotenoid biosynthesis, refers to an enzyme that catalyzes the reversible reaction of the following Reaction Scheme, and specifically, it may refer to an enzyme that synthesizes phytoene, but is not limited thereto.

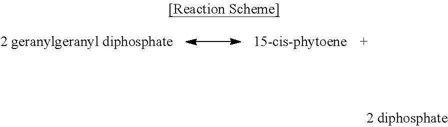

As used herein, the term “desaturase (crtl)” refers to an enzyme that catalyzes the reversible reaction of the following Reaction Scheme, and specifically, it may refer to an enzyme that synthesizes lycopene, but is not limited thereto. Further, desaturase may be used interchangeably with phytoene desaturase.

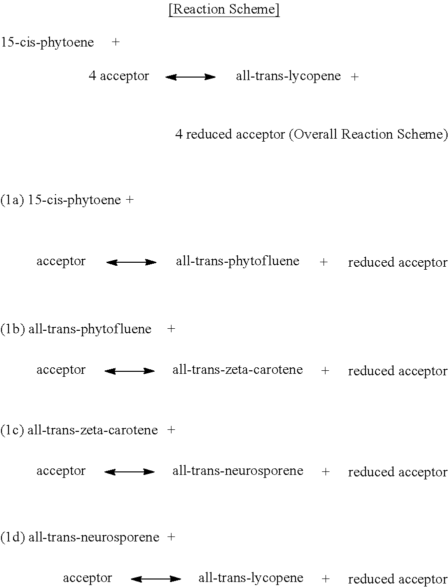

As used herein, the term “beta-carotene 15,15′-monooxygenase (β-carotene 15,15′-monooxygenase, BCMO)” refers to an enzyme that catalyzes the following Reaction Scheme, and specifically, it may refer to an enzyme that cleaves beta-carotene using oxygen molecules to produce two molecules of retinal. beta-Carotene 15,15′-monooxygenase may also process beta-cryptoxanthin, apocarotenal, 4′-apo-beta-carotenal, alpha-carotene, and gamma-carotene, and it may also be used interchangeably with beta-carotene 15,15′-dioxygenase (β-carotene 15,15′-dioxygenase).

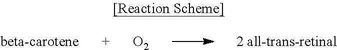

As used herein, the term “retinol dehydrogenase (ybbO)” refers to an enzyme that catalyzes the reversible reaction of the following Reaction Scheme, and all-trans- or cis-retinol may be a substrate of the enzyme. Two substrates (retinol and NAD⁺) may be used to produce three products (retinal, NADH, and H⁺). It may also be used interchangeably with microsomal retinol dehydrogenase, all-trans-retinol dehydrogenase, retinal reductase, and retinene reductase.

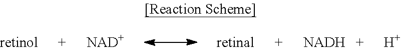

The genetic information of geranylgeranyl diphosphate synthase, phytoene synthase, desaturase, beta-carotene 15,15′-monooxygenase, and retinol dehydrogenase may be obtained from a public database, for example, GenBank of the National Center for Biotechnology Information (NCBI), etc., but is not limited thereto.

The geranylgeranyl diphosphate synthase, phytoene synthase, and desaturase may be derived from Xanthophyllomyces dendrorhous, and the beta-carotene 15,15′-monooxygenase and retinol dehydrogenase may be derived from Halobacterium sp. NRC-1, marine bacterium 66A03, or Escherichia sp. microorganisms, but they are not limited to the origin or sequence thereof, because there may be differences in the amino acid sequence of the active protein depending on the species of a microorganism or the microorganism. In particular, the retinol dehydrogenase may be derived from an Escherichia sp. microorganism, specifically, E. coli, but is not limited thereto.

Specifically, the geranylgeranyl diphosphate synthase may be a protein including an amino acid sequence of SEQ ID NO: 7, the phytoene synthase may be a protein including an amino acid sequence of SEQ ID NO: 8, and the desaturase may be a protein including an amino acid sequence of SEQ ID NO: 9. With regard to the beta-carotene 15,15′-monooxygenase, BCMO-SR may be a protein including an amino acid sequence of SEQ ID NO: 10, and BCMO-blh may be a protein including an amino acid sequence of SEQ ID NO: 11. The retinol dehydrogenase may be a protein including an amino acid sequence of SEQ ID NO: 12. However, these are not limited thereto. In the present invention, “protein including an amino acid sequence” may be used interchangeably with “protein consisting of an amino acid sequence”.

Further, in the present invention, each of the enzymes may include a protein exhibiting 80% or more, 85% or more, 90% or more, 91% or more, 92% or more, 93% or more, 94% or more, 95% or more, 96% or more, 97% or more, 98% or more, or 99% or more homology to the amino acid sequence, as well as the above-described SEQ ID NO, as long as it has a biological activity identical or corresponding to that of each enzyme.

Further, it is obvious that any amino acid sequence with deletion, modification, substitution, or addition in part of the sequence may also be included within the scope of the present disclosure, as long as the amino acid sequence has homology to the above sequence and has a biological activity substantially identical or corresponding to that of the enzyme protein of the above-described SEQ ID NO.

As used herein, the term “homology” refers to a degree of matching with a given amino acid sequence or nucleotide sequence, and the homology may be expressed as a percentage. In the present specification, a homology of a sequence having an activity which is identical or similar to that of a given amino acid sequence or nucleotide sequence is expressed as “% homology”. The homology of a sequence may be determined by, for example, a standard software, specifically BLAST 2.0, which calculates the parameters such as score, identity, similarity, etc., or by comparing the sequences in a Southern hybridization experiment under defined stringent conditions, and defining appropriate hybridization conditions is within the skill of the art, and may be determined using a method well known to those skilled in the art (e.g., J. Sambrook et al., Molecular Cloning, A Laboratory Manual, 2nd Edition, Cold Spring Harbor Laboratory press, Cold Spring Harbor, N.Y., 1989: F. M. Ausubel et al., Current Protocols in Molecular Biology, John Wiley & Sons, Inc., New York). The term “stringent conditions” refers to conditions which allow specific hybridization between polynucleotides. For example, such conditions are disclosed in detail in the literature (e.g., J. Sambrook et al., supra).

Each of the geranylgeranyl diphosphate synthase, phytoene synthase, desaturase, beta-carotene 15,15′-monooxygenase, and retinol dehydrogenase of the present invention may include a polynucleotide encoding the amino acid sequence of the above-described SEQ ID NO, or a protein exhibiting 80% or more, 85% or more, 90% or more, 91% or more, 92% or more, 93% or more, 94% or more, 95% or more, 96% or more, 97% or more, 98% or more, or 99% or more homology to the sequence, as long as it has a biological activity identical or corresponding to that of each enzyme. Specifically, a gene encoding the geranylgeranyl diphosphate synthase (GGPP synthase, crtE) may include a nucleotide sequence of SEQ ID NO: 1; a gene encoding the phytoene synthase (crtYB) may include a nucleotide sequence of SEQ ID NO: 2; a gene encoding the desaturase (crtl) may include a nucleotide sequence of SEQ ID NO: 3; with regard to genes encoding the beta-carotene 15,15′-monooxygenase (BCMO), a BCMO-SR gene may include a nucleotide sequence of SEQ ID NO: 4, and a BCMO-blh gene may include a nucleotide sequence of SEQ ID NO: 5; and a gene encoding the retinol dehydrogenase may include a nucleotide sequence of SEQ ID NO: 6.

Further, each of the polynucleotides encoding the enzymes may undergo various modifications in the coding region without changing the amino acid sequence of the protein expressed from the coding region, due to codon degeneracy or in consideration of the codons preferred in an organism in which the protein is to be expressed. Therefore, each of the polynucleotides may include any polynucleotide sequence without limitation, as long as it encodes each enzyme protein.

Further, a probe which may be prepared from a known gene sequence, for example, any polynucleotide sequence which hybridizes with a sequence complementary to all or a part of the polynucleotide sequence under stringent conditions to encode the protein having activity of geranylgeranyl diphosphate synthase, phytoene synthase, desaturase, beta-carotene 15,15′-monooxygenase, or retinol dehydrogenase enzyme, may be included without limitation.

The “stringent conditions” refer to conditions which allow specific hybridization between polynucleotides. Such conditions are disclosed in detail in the literature (e.g., J. Sambrook et al., supra). For example, the stringent conditions may include conditions under which genes having high homology, genes having 40% or more, specifically 90% or more, more specifically 95% or more, much more specifically 97% or more, and particularly specifically 99% or more homology hybridize with each other, while genes having homology lower than the above homology do not hybridize with each other; or may include ordinary washing conditions of Southern hybridization, i.e., washing once, specifically twice or three times, at a salt concentration and a temperature corresponding to 60° C., 1×SSC, 0.1% SDS; specifically 60° C., 0.1×SSC, 0.1% SDS; and more specifically 68° C., 0.1×SSC, 0.1% SSD.

Hybridization requires that two polynucleotides have complementary sequences, although mismatches between bases are possible depending on the stringency of the hybridization. The term “complementary” is used to describe a relationship between nucleotide bases that may hybridize with each other. For example, with respect to DNA, adenosine is complementary to thymine, and cytosine is complementary to guanine. Therefore, the present disclosure may also include an isolated polynucleotide fragment complementary to the entire sequence as well as a polynucleotide sequence substantially similar thereto.

Specifically, a polynucleotide having homology may be detected using hybridization conditions including a hybridization step at a Tm value of 55° C. under the above-described conditions. Additionally, the Tm value may be 60° C., 63° C., or 65° C., but is not limited thereto, and may be appropriately controlled by those skilled in the art depending on the purpose thereof.

The appropriate stringency for hybridizing polynucleotides depends on the length and degree of complementarity of the polynucleotides, and these variables are well known in the art (see Sambrook et al., supra, 9.50-9.51, 11.7-11.8).

As used herein, the term “enhancement of activity” means that activity of the enzyme protein is introduced, or the activity is increased, as compared with endogenous activity of a microorganism or activity before modification. The “introduction” of the activity means that activity of a specific protein that is not originally possessed by a microorganism is naturally or artificially exhibited. Specifically, the microorganism, in which activity of the enzyme protein is enhanced, refers to a microorganism, in which activity of the enzyme protein is enhanced, as compared with that of the natural wild-type microorganism or non-transformed microorganism. The enhancement of the activity may include, for example, enhancement by introducing foreign geranylgeranyl diphosphate synthase, phytoene synthase, desaturase, beta-carotene 15,15′-monooxygenase, and/or retinol dehydrogenase; or enhancement of the activity of endogenous geranylgeranyl diphosphate synthase, phytoene synthase, desaturase, beta-carotene 15,15′-monooxygenase, and/or retinol dehydrogenase, and specifically, the method of enhancing the activity in the present invention may be, for example, performed by way of:

1) a method of increasing the copy number of the polynucleotide encoding each enzyme,

2) a method of modifying an expression regulatory sequence to increase expression of each polynucleotide,

3) a method of modifying the polynucleotide sequence on the chromosome to enhance the activity of each enzyme, or

4) a combination of the methods, but the method is not limited thereto.

1) The method of increasing the copy number of the polynucleotide may be performed, but is not particularly limited to, in the form in which the polynucleotide is operably linked to a vector; or by integrating the polynucleotide into the chromosome of a host cell. In addition, the increase in the copy number may be performed by introducing, into a host cell, a foreign polynucleotide exhibiting the activity of the enzyme or a variant polynucleotide obtained by codon-optimizing the polynucleotide. The foreign polynucleotide may be used without limitation in the origin or sequence thereof, as long as it exhibits activity identical/similar to that of the enzyme. The introducing may be performed using a known transformation method which is appropriately selected by those skilled in the art. When the introduced polynucleotide is expressed in the host cell, the enzyme is produced, and thus its activity may be increased. Next, 2) the method of modifying an expression regulatory sequence to increase expression of each polynucleotide may be performed, but is not particularly limited to, by inducing a variation in the nucleotide sequence via deletion, insertion, non-conservative or conservative substitution, or any combination thereof so as to further enhance the activity of the expression regulatory sequence, or by replacing the sequence with a nucleotide sequence having a stronger activity. The expression regulatory sequence may include, but is not particularly limited to, a promoter, an operator sequence, a sequence encoding a ribosome-binding site, a sequence for regulating termination of transcription and translation, etc.

Specifically, a strong heterologous promoter, instead of the original promoter, may be linked upstream of the polynucleotide expression unit, and examples of the strong promoter may include a CJ7 promoter, a lysCP1 promoter, an EF-Tu promoter, a groEL promoter, an aceA or aceB promoter, etc. More specifically, a hxpr2 promoter or a UAS1B promoter is operably linked to improve the expression rate of the polynucleotide encoding each enzyme, but is not limited thereto.

Furthermore, 3) the method of modifying the polynucleotide sequence on the chromosome may be performed, but is not particularly limited to, by inducing a variation in the expression regulatory sequence by deletion, insertion, non-conservative or conservative substitution, or any combination thereof to further enhance the activity of the polynucleotide sequence, or by replacing the sequence with a polynucleotide sequence which is modified to have a stronger activity.

Finally, 4) the method of enhancing the activity by any combination of 1) to 3) may be performed by combining one or more of the method of increasing the copy number of the polynucleotide encoding each enzyme, the method of modifying an expression regulatory sequence to increase expression thereof, and the method of modifying the polynucleotide sequence on the chromosome, and the method of modifying the foreign polynucleotide having the activity of the enzyme or a codon-optimized variant polynucleotide thereof.

The polynucleotide may be described as a gene in the case where it refers to an assemblage of polynucleotides capable of carrying out functions. In the present disclosure, polynucleotides and genes may be used interchangeably, and polynucleotide sequences and nucleotide sequences may be used interchangeably.

As used herein, the term “vector” refers to a DNA construct containing a target protein-encoding polynucleotide sequence operably linked to a suitable regulatory sequence so as to be able to express the target protein in a suitable host cell. The regulatory sequence may include a promoter capable of initiating transcription, any operator sequence for regulating the transcription, a sequence encoding a suitable mRNA ribosome binding site, and a sequence for regulating termination of transcription and translation. Once transformed into an appropriate host cell, the vector may replicate or function independently from the host genome, or may integrate into a genome thereof.

The vector used in the present disclosure is not particularly limited, as long as it may replicate in a host cell. Any vector known in the art may be used. Examples of the vectors commonly used may include natural or recombinant plasmids, cosmids, viruses, and bacteriophages. For example, pWE15, M13, MBL3, MBL4, IXII, ASHII, APII, t10, t11, Charon4A, Charon21A, etc. may be used as a phage vector or cosmid vector. pBR-based, pUC-based, pBluescriptII-based, pGEM-based, pTZ-based, pCL-based, pET-based vectors, etc. may be used as a plasmid vector. Specifically, pDZ, pACYC177. pACYC184, pCL, pECCG117, pUC19, pBR322. pMW118, pCC1BAC, pSKH, pRS-413, pRS-414, pRS-415, pBCA, or pYLI vector, etc. may be used, but the vector is not limited thereto.

The vector applicable in the present invention is not particularly limited, and a known expression vector may be used. Additionally, a polynucleotide encoding a target protein may be integrated into the chromosome through a vector for chromosomal insertion. The integration of the polynucleotide into the chromosome may be performed using any method known in the art, for example, by homologous recombination, but is not limited thereto. A selection marker for confirming the integration into the chromosome may be further included, or a gene related thereto may be removed. The selection marker is used for selecting a cell transformed with a vector, i.e., in order to confirm whether or not the target nucleic acid molecules have been inserted, and markers that provide selectable phenotypes, such as drug resistance, auxotrophy, resistance to cytotoxic agents, or expression of surface proteins. may be used. Only the cells expressing the selection markers are able to survive or express other phenotypic traits under the environment treated with selective agents, and therefore, the transformed cells may be selected.

As used herein, the term “transformation” means introduction of a vector including a polynucleotide encoding a target protein into a host cell in such a way that the protein encoded by the polynucleotide is expressed in the host cell. As long as the transformed polynucleotide may be expressed in the host cell, it may be integrated into and located in the chromosome of the host cell or exist extrachromosomally, or it may be in any of the cases. Further, the polynucleotide includes DNA and RNA which encode the target protein. The polynucleotide may be introduced in any form as long as it may be introduced into a host cell and expressed therein. For example, the polynucleotide may be introduced into a host cell in the form of an expression cassette, which is a gene construct including all elements necessary for autonomous expression. The expression cassette may commonly include a promoter operably linked to the polynucleotide, a transcription termination signal, a ribosome binding site, and a translation termination signal. The expression cassette may be in the form of an expression vector capable of self-replication. Further, the polynucleotide may be introduced into a host cell as it is and operably linked to a sequence necessary for its expression in the host cell, but is not limited thereto. The transformation method includes any method of introducing a nucleic acid into a cell, and may be performed by selecting a suitable standard technique known in the art according to the host cell. For example, the transformation may be carried out via electroporation, calcium phosphate (CaPO₄) precipitation, calcium chloride (CaCl₂) precipitation, microinjection, a polyethylene glycol (PEG) technique, a DEAE-dextran technique, a cationic liposome technique, a lithium acetate-DMSO technique, etc., but the method is not limited thereto.

Further, the term “operably linked” means a functional linkage between a promoter sequence which initiates and mediates transcription of the polynucleotide encoding the target protein of the present disclosure and the polynucleotide sequence. The operable linkage may be prepared using a genetic recombinant technique known in the art, and site-specific DNA cleavage and ligation may be performed using enzymes, such as restriction enzymes, ligases, etc., known in the art, but is not limited thereto.

The microorganism of the present invention may be a microorganism in which Ku70 or Ku80 protein activity is inactivated or activity of URA3 gene is inactivated.

As used herein, the term “Ku70” or “Ku80” refers to a Ku protein, and Ku70 and Ku80 form a Ku heterodimer, which binds to the ends of a DNA double-strand break, and is involved in DNA repair via non-homologous end joining (NHEJ). In the present invention, to increase the efficiency of homologous recombination of the retinol biosynthetic genes, a microorganism was used in which one of Ku70/Ku80 genes having a function of inhibiting random insertion was deleted.

As used herein, the term “URA3 gene” refers to a gene encoding orotidine 5′-phosphate decarboxylase (ODCase) in a pyrimidine synthetic pathway of a yeast. Further, URA3+ yeast is characterized in that it may proliferate in a medium to which uracil or uridine is not added, but it does not proliferate in a medium containing 5-fluoroorotic acid (5-FOA), which is a pyrimidine analog. URA3 gene may be used as a positive or negative marker by employing the above characteristics.

According to one exemplary embodiment of the present invention, to examine whether or not the retinol biosynthetic genes were introduced, a URA3 gene-deleted yeast was prepared. A method of introducing the URA3 gene together, when the retinol biosynthetic genes were introduced, was used to select a strain in which the retinol biosynthetic genes were introduced in a medium to which uracil or uridine was not added (Example 1-1).

As used herein, the term “inactivation” refers to a case where the activity of an enzyme protein originally possessed by a microorganism is weakened, as compared to the endogenous activity thereof or the activity before modification; a case where no protein is expressed at all; or a case where the protein is expressed but exhibits no activity. The inactivation is a concept that includes a case where the activity of an enzyme itself is weakened or eliminated, as compared to the activity of the enzyme originally possessed by a microorganism, due to a modification in the polynucleotide encoding the enzyme, etc.; a case where the level of overall enzyme activity within a cell is reduced or eliminated, as compared to the wild-type microorganism, due to inhibition of expression of the gene encoding the enzyme, or inhibition of translation, etc.; a case where a part or the entirety of the gene encoding the enzyme is deleted; and a combination thereof, but is not limited thereto. In other words, the microorganism in which activity of the enzyme protein is inactivated refers to a microorganism in which the activity of the enzyme protein is reduced or eliminated, as compared to the natural wild-type microorganism or the non-modified microorganism.

The inactivation of the enzyme activity may be achieved by way of various methods well known in the art. Examples of the methods include 1) a method of deleting a part or the entirety of the gene encoding the enzyme on the chromosome; 2) a method of modifying an expression regulatory sequence such that the expression of the gene encoding the protein on the chromosome is reduced; 3) a method of modifying a gene sequence encoding the protein on the chromosome such that the activity of the protein is removed or weakened; 4) a method of introducing an antisense oligonucleotide (e.g., antisense RNA) that binds complementarily to the transcript of the gene encoding the protein on the chromosome; 5) a method of adding a sequence complementary to the Shine-Dalgamo sequence of the gene encoding the protein on the chromosome upstream of the Shine-Dalgamo sequence to form a secondary structure, thereby making the adhesion of ribosome impossible: and 6) a method of reverse transcription engineering (RTE), which adds a promoter, which is to be reverse-transcribed, to the 3′-end of the open reading frame (ORF) of the polynucleotide sequence encoding the protein, or a combination thereof, but are not limited particularly thereto.

The method of deleting a part or the entirety of the gene encoding the enzyme on the chromosome may be performed by replacing the polynucleotide encoding the endogenous target protein within the chromosome with a polynucleotide having a partially deleted nucleic acid sequence, or a marker gene through a vector for chromosomal insertion into a microorganism. As an example of the method of deleting a part or the entirety of the polynucleotide, a method of deleting the polynucleotide by homologous recombination may be used, but is not limited thereto.

The method of modifying the expression regulatory sequence may be performed by inducing a modification in the expression regulatory sequence through deletion, insertion, non-conservative or conservative substitution, or a combination thereof so as to further weaken the activity of the expression regulatory sequence: or by replacing the sequence with a nucleic acid sequence having a weaker activity. The expression regulatory sequence may include a promoter, an operator sequence, a sequence encoding a ribosome binding site, and a sequence for regulating the termination of transcription and translation, but is not limited thereto.

The method of modifying the gene sequence on the chromosome may be performed by inducing a modification in the gene sequence through deletion, insertion, non-conservative or conservative substitution, or a combination thereof so as to further weaken the activity of the enzyme: or by replacing the sequence with a gene sequence modified to have a weaker activity or a gene sequence modified to have no activity at all, but is not limited thereto.

As used herein, the term “part”, although it may vary depending on the kinds of polynucleotide, may specifically refer to 1 to 300 nucleotides, more specifically 1 to 100 nucleotides, and even more specifically 1 to 50 nucleotides, but is not particularly limited thereto.

The microorganism for producing retinol may be a microorganism in which geranylgeranyl diphosphate synthase (GGPP synthase, crtE), phytoene synthase (crtYB), and desaturase (crtl) protein activities are enhanced, and may be a microorganism capable of producing beta-carotene and having more improved ability to produce retinol by further enhancing beta-carotene 15,15′-monooxygenase and retinol dehydrogenase protein activities.

The microorganism of the present invention is not limited, as long as it is a microorganism having an ability to produce retinol, and specifically, the microorganism may be Yarrowia lipolytica or Saccharomyces cerevisiae.

Another aspect of the present invention provides a method of producing retinol, the method including the step of culturing the microorganism of the present invention in a culture medium.

The “microorganism” and “retinol” are the same as described above.

As used herein, the term “culturing” refers to growing the microorganism in an appropriately controlled environment. The culture process of the present invention may be achieved according to appropriate medium and culture conditions known in the art. The culture process may be easily adjusted for use by those skilled in the art according to the microorganism to be selected. Although not particularly limited thereto, the step of culturing the microorganism may be performed by a known batch culture method, continuous culture method, fed-batch culture method, etc. The medium used for culturing the microorganism of the present disclosure and other culture conditions are not particularly limited, but any medium commonly used for culturing the microorganism may be used. Specifically, the microorganism of the present disclosure may be cultured in a common medium containing suitable carbon sources, nitrogen sources, phosphorous sources, inorganic compounds, amino acids, and/or vitamins, etc., under aerobic conditions while controlling temperature, pH, etc.

The pH in the step of culturing may be adjusted using a basic compound (e.g., sodium hydroxide, potassium hydroxide, or ammonia) or an acidic compound (e.g., phosphoric acid or sulfuric acid), and specifically, the pH may be 5.5 to 7.5, 5.5 to 7.0, 6.0 to 7.5, 6.0 to 7.0, 6.5 to 7.5, or 6.5 to 7.0, and more specifically, the pH in the step of culturing may be 6.9, but is not limited thereto.

Further, oxygen or oxygen-containing gas may be injected into the culture in order to maintain an aerobic state of the culture; or no gas may be injected. or nitrogen gas, hydrogen gas, or carbon dioxide gas may be injected to maintain an anaerobic or microaerobic state. The aeration rate in the step of culturing may be 0.2 vvm to 1.5 vvm, 0.2 vvm to 1.3 vvm, 0.2 vvm to 1.1 vvm, 0.5 vvm to 1.5 vvm, 0.5 vvm to 1.3 vvm, 0.5 vvm to 1.1 vvm, 0.8 vvm to 1.5 vvm, 0.8 vvm to 1.3 vvm, 0.8 vvm to 1.1 vvm, and more specifically, the aeration rate in the step of culturing may be 0.9 vvm, but is not limited thereto.

Further, the culture temperature may be maintained at 20° C. to 45° C., or 25° C. to 40° C., specifically at 27° C. to 31° C., 28° C. to 31° C., 29° C. to 31° C., or 30° C. to 31° C., and more specifically at 30.2° C., but is not limited thereto.

Further, the reactor operating rpm in the step of culturing may be 50 rpm to 300 rpm, 50 rpm to 250 rpm, 100 rpm to 300 rpm, 100 rpm to 250 rpm, 150 rpm to 300 rpm, 150 rpm to 250 rpm, 200 rpm to 300 rpm, or 200 rpm to 250 rpm, and more specifically 249.9 rpm, but is not limited thereto.

As used herein, the term “medium” refers to a culture medium for culturing the microorganism of the present invention and/or a product obtained after culturing the same. The medium is a concept that includes both a form containing the microorganism and a form in which the microorganism is removed by centrifugation, filtration, etc. from the culture solution containing the microorganism.

In the culture medium used for culturing the microorganism for producing retinol of the present invention, as a carbon source, sugars and carbohydrates (e.g., glucose, sucrose, lactose, fructose, maltose, molasses, starch, and cellulose), oils and fats (e.g., soybean oil, sunflower seed oil, peanut oil, and coconut oil), fatty acids (e.g., palmitic acid, stearic acid, and linoleic acid), alcohols (e.g., glycerol and ethanol), organic acids (e.g., acetic acid), etc. may be used alone or in combination, but the carbon source is not limited thereto. As a nitrogen source, nitrogen-containing organic compounds (e.g., peptone, yeast extract, meat gravy, malt extract, corn steep liquor, soybean flour, and urea) or inorganic compounds (e.g., ammonium sulfate, ammonium chloride, ammonium phosphate, ammonium carbonate, and ammonium nitrate), etc. may be used alone or in combination, but the nitrogen source is not limited thereto. As a phosphorus source, potassium dihydrogen phosphate, dipotassium hydrogen phosphate, corresponding sodium-containing salts thereof, etc. may be used alone or in combination, but the phosphorus source is not limited thereto. Further, essential growth-promoting materials such as other metal salts (e.g., magnesium sulfate or iron sulfate), amino acids, vitamins, etc. may be included in the medium.

The culture medium used for culturing the microorganism for producing retinol of the present invention may include one or more nutrients selected from the group consisting of yeast extract, peptone, soybean, and glucose.

The yeast extract may be included in an appropriate amount in the culture medium for the microorganism for producing retinol of the present invention, specifically in an amount of 1 part by weight to 4 parts by weight, 1.5 parts by weight to 4 parts by weight, 2 parts by weight to 4 parts by weight, 2.5 parts by weight to 4 parts by weight, 1 part by weight to 3.5 parts by weight, 1.5 parts by weight to 3.5 parts by weight, 2 parts by weight to 3.5 parts by weight, or 2.5 parts by weight to 3.5 parts by weight, and more specifically in an amount of 3 parts by weight, based on the total 100 parts by weight of the culture medium, but is not limited thereto.

Further, the peptone may be included in an appropriate amount in the culture medium for the microorganism for producing retinol of the present invention, specifically in an amount of 0.5 parts by weight to 4 parts by weight, 1 part by weight to 4 parts by weight, 1.5 parts by weight to 4 parts by weight, 2 parts by weight to 4 parts by weight, 2.5 parts by weight to 4 parts by weight, 0.5 parts by weight to 4 parts by weight, 1 part by weight to 3.5 parts by weight, 1.5 parts by weight to 3.5 parts by weight, 2 parts by weight to 3.5 parts by weight, or 2.5 parts by weight to 3.5 parts by weight, and more specifically in an amount of 3 parts by weight, based on the total 100 parts by weight of the culture medium, but is not limited thereto.

Further, the soybean may be included in an appropriate amount in the culture medium for the microorganism for producing retinol of the present invention, specifically in an amount of 0.5 parts by weight to 4 parts by weight, 1 part by weight to 4 parts by weight, 1.5 parts by weight to 4 parts by weight, 2 parts by weight to 4 parts by weight, 2.5 parts by weight to 4 parts by weight, 0.5 parts by weight to 4 parts by weight, 1 part by weight to 3.5 parts by weight, 1.5 parts by weight to 3.5 parts by weight, 2 parts by weight to 3.5 parts by weight, or 2.5 parts by weight to 3.5 parts by weight, and more specifically in an amount of 3 parts by weight, based on the total 100 parts by weight of the culture medium, but is not limited thereto.

Further, the glucose may be included in an appropriate amount in the culture medium for the microorganism for producing retinol of the present invention, specifically in an amount of 1 part by weight to 3 parts by weight, 1 part by weight to 2.5 parts by weight, 1.5 parts by weight to 3 parts by weight, or 1.5 parts by weight to 2.5 parts by weight, and more specifically in an amount of 2 parts by weight, based on the total 100 parts by weight of the culture medium, but is not limited thereto.

The retinol produced by the culture may be secreted into the medium or may remain in the cells.

Further, the method of producing retinol of the present invention may be a method of using a multi-copy plasmid of the microorganism. Specifically, in one exemplary embodiment, it was confirmed that the retinol production was increased about 3-fold in a strain transformed with the multi-copy plasmid, as compared with a strain transformed with a single-copy plasmid (Example 3-2).

Further, the method of producing retinol of the present invention may further include the step of recovering retinol from the cultured microorganism or medium. In the step of recovering retinol produced in the culturing step of the present invention, the desired retinol may be collected from the microorganism for producing retinol and the culture solution thereof using an appropriate method known in the art according to the culture method. For example, lyophilization, centrifugation, filtration, anion-exchange chromatography, crystallization, HPLC, etc. may be used, and the desired retinol may be recovered from the cultured microorganism or the medium using an appropriate method known in the art. The step of recovering retinol may further include a separation process and/or a purification step.

Hereinafter, the present invention will be described in more detail with reference to Examples. However, these Examples are for illustrative purposes only, and the scope of the present invention is not intended to be limited by these Examples.

### Example 1: Selection of Strain for Producing Retinol and Gene Synthesis Through Codon Optimization

### Example 1-1: Preparation of Saccharomyces cerevisiae Platform Strain for Producing Retinol

Saccharomyces cerevisiae, which is a yeast strain widely used in recombinant protein production, was selected as a strain for producing retinol.

Next, to prepare the strain for producing retinol, high-efficiency gene manipulation in the genome of a Saccharomyces cerevisiae strain is required, and to this end, it is necessary to establish a high-efficiency gene manipulation system capable of repeatedly integrating or removing a specific gene group in the genome, and a system capable of performing a homologous recombination of the integrated gene within the genome with high efficiency. Therefore, a Saccharomyces cerevisiae platform strain was prepared in which the above system was established.

In detail, to increase efficiency of homologous recombination, one of Ku70/Ku80 genes having the function of inhibiting random insertion should be removed, and to use URA3 as an auxotrophic selection marker for confirming foreign gene integration, URA3 gene in the genome should be removed. Accordingly, a Saccharomyces cerevisiae platform strain for producing retinol was prepared in which URA3 and Ku70 were removed. This strain was used in the following experiments.

### Example 1-2: Design of Saccharomyces cerevisiae Customized High-Efficiency Retinol Production Metabolic Pathways and Gene Synthesis

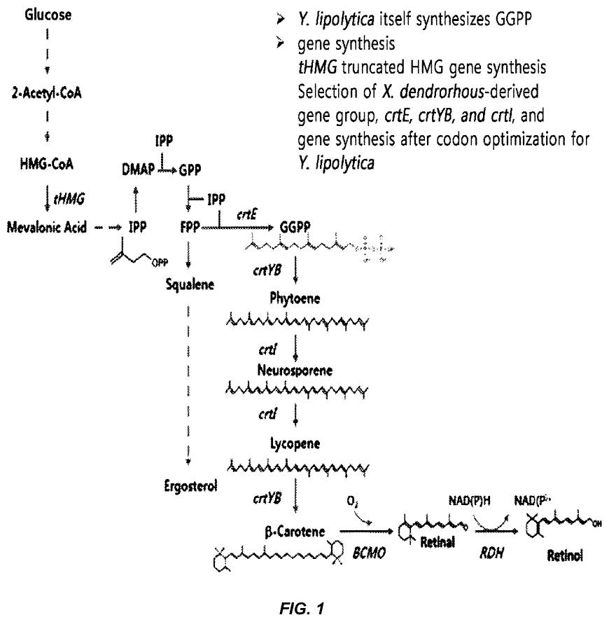

As confirmed in FIG. 1, metabolic pathways from GGPP production to retinol production (GGPP→phytoene→neurosporene→lycopene→β-carotene→retinal→retinol) were designed.

In detail, geranylgeranyl diphosphate synthase (GGPP synthase), phytoene synthase, and desaturase-encoding nucleotide sequences (crtE, crtl, and crtYB. respectively), which are Xanthophyllomyces dendrorhous-derived beta-carotene biosynthetic genes, were codon-optimized for Saccharomyces cerevisiae, and then each gene was synthesized; a beta-carotene 15,15′-monooxygenase (β-carotene 15,15′-monooxygenase)-encoding nucleotide sequence (BMCO-blh or BMCO-SR), which is Halobacterium sp. NRC-1- or marine bacterium 66A03-derived retinal biosynthetic gene, was codon-optimized for Saccharomyces cerevisiae, and then each gene was synthesized; and a retinol dehydrogenase-encoding nucleotide sequence (ybbO), which is a retinol biosynthetic gene, was codon-optimized for Saccharomyces cerevisiae, and then the gene was synthesized.

The nucleotide sequences encoding crtE, crtYB, crtl, BMCO-blh, BMCO-SR, and ybbO are represented by SEQ ID NO: 1, SEQ ID NO: 2, SEQ ID NO: 3, SEQ ID NO: 4, SEQ ID NO: 5, and SEQ ID NO: 6, respectively.

### Example 2: Preparation of Strain for Producing Retinol

### Example 2-1: Preparation of Expression Vector for S. cerevisiae for Producing Retinol

To stably express crtE. crtl, crtYB, BMCO-blh, BMCO-SR, and ybbO gene nucleotide sequences synthesized in Example 1-2 in the Saccharomyces cerevisiae platform strain for producing retinol, an expression vector was prepared in which a GPD promoter and a CYC1 terminator were included and a ura marker system for repeated use of the vector system was applied.

In detail, crtE, crtl, crtYB, BMCO-blh, BMCO-SR, and ybbO gene-encoding nucleotide sequences synthesized in Example 1-2 were inserted into the expression vector, into which the GPD promoter, the CYC1 terminator, and the URA Blaster cassette were introduced, using EcRI and XhoI restriction enzymes within the multi cloning site, respectively. pBCA_crtE, pBCA crti, pBCA_crtYB, pRET_BMCO-blh, pRET_BMCO-SR, and pRET_ybbO vectors, into which each gene sequence was inserted, were prepared, respectively.

### Example 2-2: Integration of Retinol Precursor Biosynthetic Genes

As integration sites in the genome of S. cerevisiae, a YPL062w gene known to increase production of mevalonate, which is a precursor of retinol, when removed, and LPP1 and DPP1 genes producing famesol, which are involved in famesyl pyrophosphate (FPP) depletion, were selected in order to increase supply of the retinol precursor.

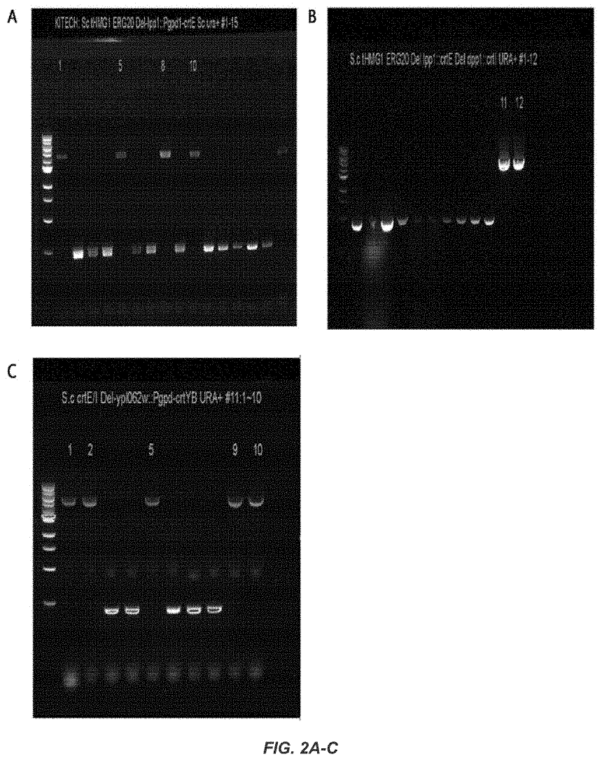

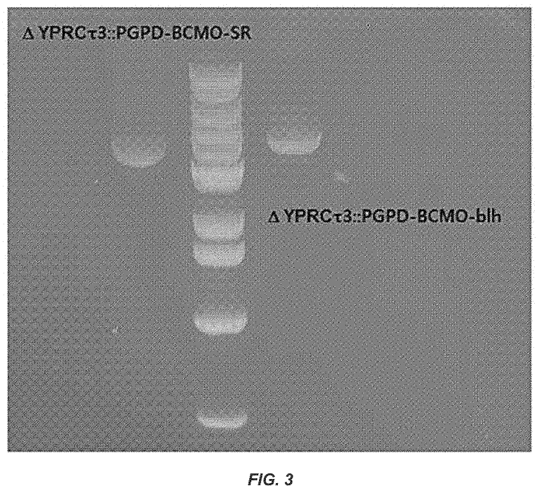

In detail, the Saccharomyces cerevisiae platform strain for producing retinol, which was prepared in Example 1-1, was transformed with the pBCA_crtE vector prepared in Example 2-1, and then it was examined through colony PCR whether or not the crtE gene was integrated at the correct site in the strain (FIG. 2A). When crtE gene was integrated at the correct site, bands of 4,000 bp were observed, as in lanes 1, 5, 8, and 10 of FIG. 3A. The ura marker was removed from the strain, in which the integration was confirmed, through ura pop-out, and then used in the subsequent crtl gene integration.

The method above was used to integrate crtl gene and crtYB gene sequentially, and the integration of the genes was examined (FIGS. 2B and 2C). When crtl gene is integrated at the correct site, bands appear around 4.000 bp, as in lanes 11 and 12 of FIG. 2B. When crtYB gene is integrated at the correct site, bands appear around 6,000 bp, as in lanes 1, 2, 5, 9, and 10 of FIG. 2C. As described, the strain, in which crtE, crtl, and crtYB genes which are biosynthetic genes of beta-carotene as a retinol precursor were integrated, was prepared. In the following experiment, the strain (S. cerevisiae CEN.PK2-1D Δleu2::PTEF1-ERG20 Δhis3::PCCW12-tHMG1 ΔIpp1::PGPD-crtE Δdpp1::PGPD-crtl Δypl062w::PGPD-crtYB) was used as a basic strain to prepare a strain for producing retinol.

### Example 2-3: Integration of Retinol Biosynthetic Genes

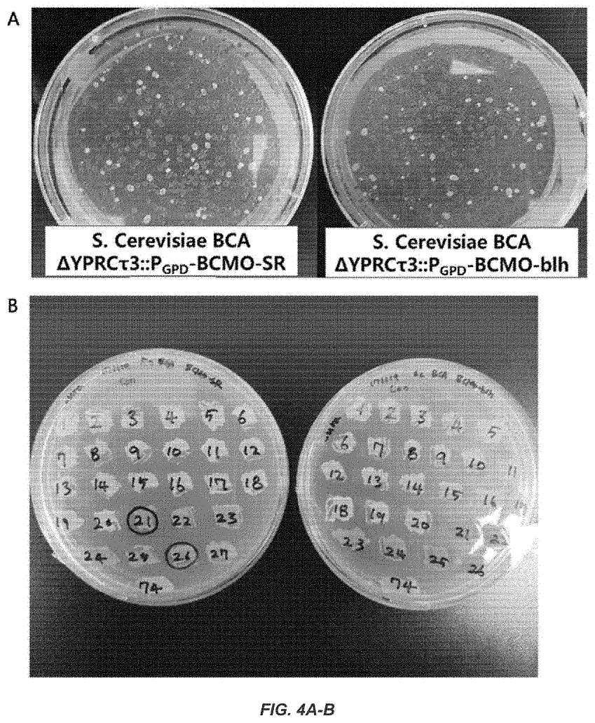

The Saccharomyces cerevisiae strain prepared in Example 2-2, in which the retinol precursor biosynthetic genes were integrated, was transformed with the pBCA-BCMO vector prepared in 2-1, and then it was examined through PCR and colony formation whether or not each of BMCO-blh or BMCO-SR genes was integrated at the correction site in the strain (FIGS. 3 and 4).

In detail, pBCA-BCMO vector and pUC-3Myc URA3-PGPD vector were treated with EcoRI/XhoI restriction enzymes to digest BCMO, followed by electrophoresis on a 1% agarose gel. The digested BCMO was purified, and the purified BCMO was ligated with the pUC-3Myc URA3-PGPD vector using EcoRI/XhoI restriction enzymes to prepare a pUC-3Myc URA3-PGPD-BCMO vector. BCMO was digested again using EcoRI/XhoI restriction enzymes. Thereafter, the BCMO gene was integrated into the YPRCT3 gene site using primers for an integration cassette, consisting of nucleotide sequences in Table 1 below.

TABLE 1
Primer sequences for BCMO gene integration
cassette

| | |
|---|---|
| Forward | ATC GTC CTT GTA TGG AAG TAT CAA AGG GGA |
| | CGT TCT TCA CCT CCT TGG AAC CAG TCA CGA |
| | CGT TGT AAA A (SEQ ID NO: 13) |
| Reverse | AAT GAT TTA CAA TCT AGT CGC AAA AAC AAG |
| | TAC AGT GCT GAC GTC CCA TCA GGT TTC CCG |
| | ACT GGA AAG C (SEQ ID NO: 14) |

As a result, as shown in FIG. 3, PCR results confirmed that a vector for PGPD-BCMO URA3 integration was prepared in which BCMO-SR or BCMO-blh gene was integrated.

Subsequently. as shown in FIGS. 4A and 4B, it was confirmed through colony formation and master colony acquisition that BMCO-blh or BMCO-SR gene was successfully transformed into the Saccharomyces cerevisiae YPRCT3 gene site by the prepared integration cassette. Subsequently, as shown in FIGS. 4C and 4D, PCR of the BCMO-integrated master colony was performed, and as a result, when BCMO-SR gene was integrated, a band appeared around 5.124 bp, as in lane 21, and when BCMO-blh gene was integrated, a band appeared around 5,124 bp, as in lanes 11 and 14. As described, a BCMO gene-introduced strain was prepared.

Finally, to prepare a strain for producing retinol, a strain for producing retinol was prepared in which the ybbO gene encoding retinol dehydrogenase was introduced into the prepared strain.

In detail, in the same manner as the method of introducing BCMO gene, a vector for pUC57-3Myc URA3-PGPD-ybbO integration was prepared. Thereafter, the ybbO gene was integrated into the YPRCdelta15 gene site using primers for an integration cassette, consisting of nucleotide sequences in Table 2 below.

TABLE 2
Primer sequences for ybbO gene integration
cassette

| | |
|---|---|
| Forward | AAATCCGAACAACAGAGCATAGGGTTTCGCAAACAAACTT |
| | AAATATATGCaggtttcccgactggaaag |
| | (SEQ ID NO: 15) |
| Reverse | GTATAATCTGTATACATAATATTATAGGCTTTACCAACAA |
| | TGGAATTTCGccagtcacgacgttgtaaaa |
| | (SEQ ID NO: 16) |

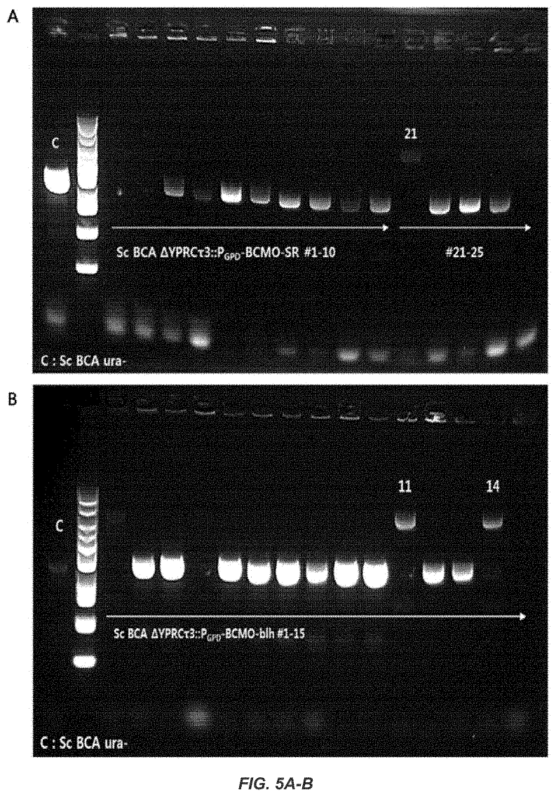

As a result, as shown in FIG. 5. PCR of the ybbO-integrated master colony was performed, and as a result, when ybbO gene was integrated, a band appeared around 3,600 bp, as in lane 11. Finally, a strain for producing retinol was prepared in which crtE, crtYB, crtl, BMCO-blh, BMCO-SR, and ybbO genes were integrated.

### Example 3: Examination of Retinol Production of Strain for Producing Retinol

### Example 3-1: Examination of Retinol Production of Strain for Producing Retinol

To examine retinol production by the strain for producing retinol, HPLC/UV chromatography and ESI-mass spectrometry were performed. In detail, chromatographic analysis was performed using an analytical scale (dimensions: 300 mm×4 mm). An ODS C18 guard column (4 mm×3 mm) preceded a running column. Chromatographic conditions were as follows: mobile phase A: acetonitrile/H₂O/glacial acetic acid=90/10/2; mobile phase B: acetonitrile/methanol=90/10; mobile phase C: 100% THF. All mobile phases were filtered through nylon membrane filters with 0.45 μm pore size before use. Over a total run time of 26 min, the following gradient program was applied at a constant flow rate (1 mL/min) and a constant column temperature (22° C.): 0-4.5 min 100% A; 4.5-6.5 min 100% B; 6.5-12 min 60% B, 40% C; 12-15 min 60% B, 40% C; 15-20 min 100% B; 20-26 min 100% A.

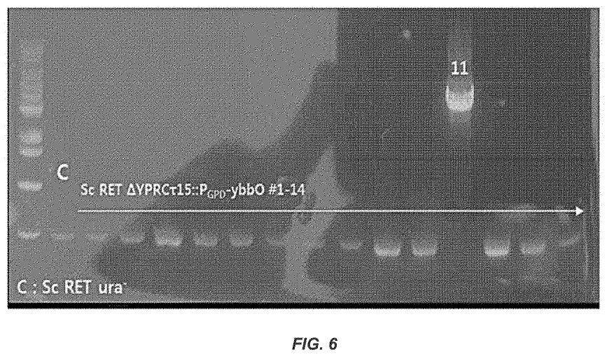

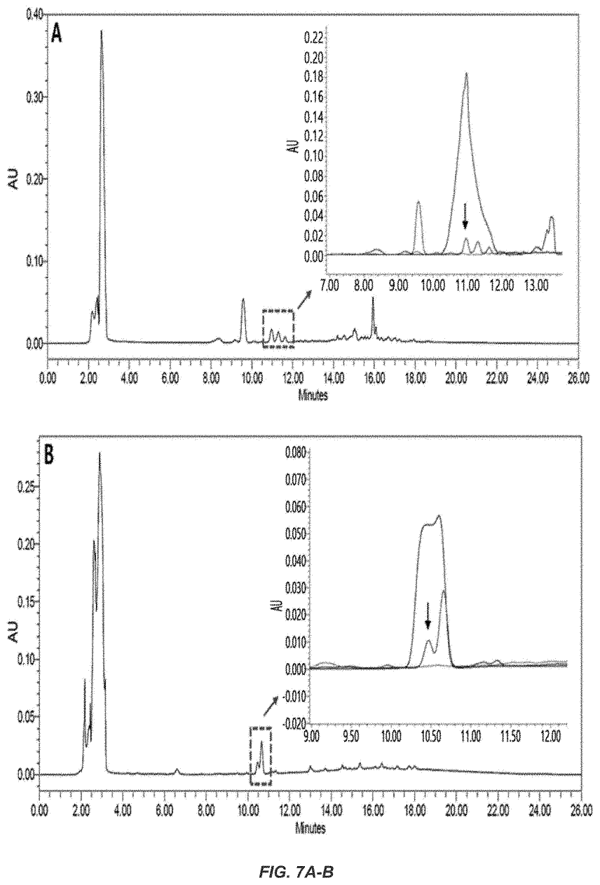

As a result, as shown in FIG. 6, a retinal production peak and a retinol production peak of the colony formed by culturing the strain for producing retinol were observed. In detail, as a result of HPLC/UV chromatography (325 nm), the retinal production peak was observed at 10.47 min (FIG. 6A), and the retinol production peak was observed at 10.97 min. Further, as shown in FIG. 7, unlike CEN.PK wild-type strain (FIG. 7A), the strain for producing retinol of the present invention showed the retinol production peak at 303.2318 m/z (FIG. 7B).

These results indicate that the strain for producing retinol prepared in Example 2 is able to produce retinol.

### Example 3-2: Examination of Effect of Increasing Retinol Production by Multi-Copy Plasmid

The effect of increasing the retinol production of the strain for producing retinol was examined when the strain transformed with a multi-copy plasmid was cultured.

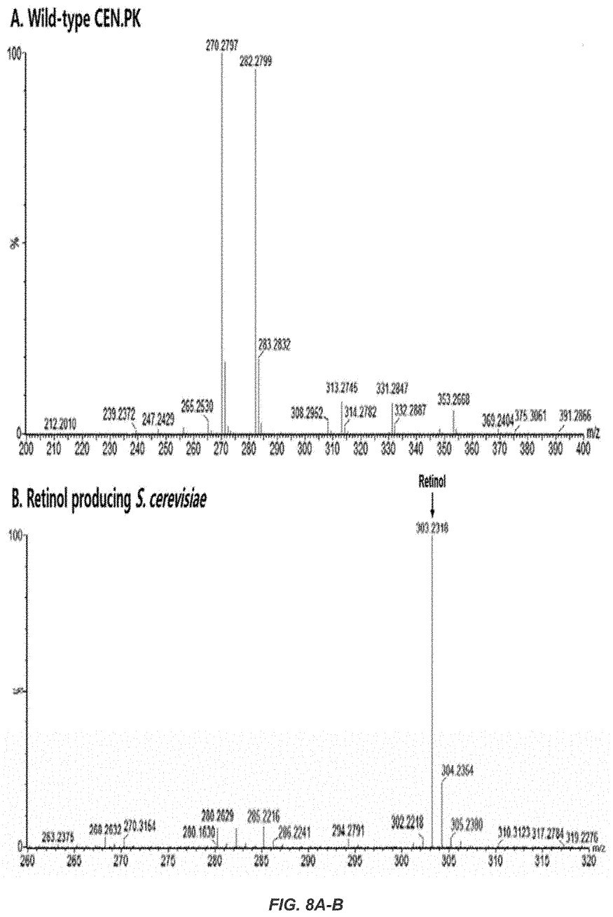

In detail, as shown in FIG. 8, when the strain was cultured by introducing the single-copy BCMO-SR gene using a pUC57 vector, it produced 169.16 μg/L of retinol. In contrast, when the strain was cultured by introducing the multi-copy BCMO-SR gene using a p426 vector, it produced 491.09 μg/L of retinol. That is, the strain transformed with the multi-copy showed an about 3-fold increase in the retinol production, as compared with the strain transformed with the single copy. These results indicate that when the microorganism transformed with the multi-copy plasmid is cultured, the retinol production may be remarkably increased.

Descriptions of contents which may be sufficiently recognized and inferred by those skilled in the art will be omitted in the present specification. In addition to the specific exemplary embodiments described in the present specification, more various modifications may be made without changing the technical spirit or essential configuration of the present invention. Accordingly, the present invention may be implemented in a manner different from those specifically described and illustrated in the present specification, which may be understood by those skilled in the art.

### Effect of the Invention

A microorganism of the present invention may have an improved ability to produce retinol, and thus it may be efficiently used in producing retinol. Based on a method of producing retinol, the method including a step of culturing the microorganism, the retinol production efficiency may be improved.

What is claimed is:

1. A microorganism for producing retinol, in which geranylgeranyl diphosphate synthase (GGPP synthase, crtE), phytoene synthase (crtYB), desaturase (crtl), beta-carotene 15,15′-monooxygenase (β-carotene 15,15′-monooxygenase, BCMO), and retinol dehydrogenase (ybbO) protein activities are enhanced.

2. The microorganism for producing retinol of claim 1, wherein the geranylgeranyl diphosphate synthase (GGPP synthase, crtE), phytoene synthase (crtYB), and desaturase (crtl) are derived from Xanthophyllomyces dendrorhous.

3. The microorganism for producing retinol of claim 1, wherein the beta-carotene 15,15′-monooxygenase (β-carotene 15,15′-monooxygenase, BCMO), and retinol dehydrogenase (ybbO) are derived from a Halobacterium sp. NRC-1, marine bacterium 66A03, or Escherichia sp. microorganism.

4. The microorganism for producing retinol of claim 1, wherein a gene of the geranylgeranyl diphosphate synthase (GGPP synthase, crtE) includes a nucleotide sequence of SEQ ID NO: 1; a gene of the phytoene synthase (crtYB) includes a nucleotide sequence of SEQ ID NO: 2; a gene of the desaturase (crtl) includes a nucleotide sequence of SEQ ID NO: 3; a gene of the beta-carotene 15,15′-monooxygenase (β-carotene 15,15′-monooxygenase, BCMO) includes a nucleotide sequence of SEQ ID NO: 4 or SEQ ID NO: 5; and a gene of the retinol dehydrogenase (ybbO) includes a nucleotide sequence of SEQ ID NO: 6.

5. The microorganism for producing retinol of claim 1, wherein the microorganism is Yarrowia lipolytica or Saccharomyces cerevisiae.

6. A method of producing retinol, the method comprising the step of culturing the microorganism of any one of claims 1 to 5 in a culture medium.

7. The method of producing retinol of claim 6, wherein the culture medium includes one or more nutrients selected from the group consisting of yeast extract, peptone, soybean, and glucose.

8. The method of producing retinol of claim 6, further comprising the step of recovering retinol from the cultured microorganism or medium.
